(19) United States
(12) Patent Application Publication
(10) Pub. No.: US 2019/0234956 A1
(43) Pub. Date: Aug. 1, 2019

(54) METHOD FOR DETECTING CROSSLINKED PEPTIDES VIA REPRODUCIBLE FRAGMENTATION IN A MASS SPECTROMETER

(71) Applicant: ARIZONA BOARD OF REGENTS ON BEHALF OF THE UNIVERSITY OF ARIZONA, Tucson, AZ (US)

(72) Inventors: William R. Montfort, Tucson, AZ (US); Jessica A. Wales, Tucson, AZ (US); Linda A. Breci, Tucson, AZ (US)

(73) Assignee: ARIZONA BOARD OF REGENTS ON BEHALF OF THE UNIVERSITY OF ARIZONA, Tucson, AZ (US)

(21) Appl. No.: 16/317,964

(22) PCT Filed: Jul. 17, 2017
(86) PCT No.: PCT/US17/42415

§ 371 Date: Jan. 15, 2019

Related U.S. Application Data
(60) Provisional application No. 62/363,144, filed on Jul. 15, 2016.

(51) Int. Cl. G01N 33/68; C07K 14/56

(52) U.S. Cl. CPC G01N 33/6845; C07K 14/56; G01N 33/6848; G01N 33/6818; G01N 33/6815

(57) ABSTRACT

Methods of identifying a crosslinking site or a binding site on a protein are described herein. The protein may comprise a binding site in a vicinity of the crosslinking site and identification of the crosslinking site may aid in identifying a location of the binding site in the protein. Methods of identifying a peptide or protein from a complex mixture are also described herein. The invention features a crosslinking agent that is configured to interact with a binding site of a protein and comprises a substituent configured to fragment in tandem mass spectrometry to yield a signature mass fragment.

### GOVERNMENT SUPPORT

This invention was made with government support under Grant No. R01 GM117357 awarded by NIH. The government has certain rights in the invention

### FIELD OF THE INVENTION

The present invention relates to a method of identifying a crosslinking site on a protein by use of tandem mass spectroscopy. The method may also be used to selectively identify a crosslinking site in the vicinity of a protein binding site and consequently help to identify the binding site.

### BACKGROUND OF THE INVENTION

Protein binding is a critical component of biochemistry. Many biological processes are mediated by noncovalent binding interactions between a protein and another molecule, its binding partner. The molecules which preferentially bind each other may be referred to as members of a “specific binding pair”. Such pairs include an antibody and its antigen, a lectin and a carbohydrate which it binds, an enzyme and its substrate, and a hormone and its cellular receptor.

A “binding site” is a point of contact between a binding surface of the binding protein and a complementary surface of the binding partner. Considerable experimental work and time are required to precisely characterize a binding site. The most definitive techniques for the characterization of the structure of receptor binding sites have been NMR spectroscopy and X-ray crystallography. While these techniques can ideally provide a precise characterization of the relevant structural features, they have major limitations, including inordinate amounts of time required for study, inability to study large proteins, and, for X-ray analysis, the need for protein-binding partner crystals.

An example of a protein with a binding site of interest is soluble guanylyl/guanylate cyclase (sGC). It is the nitric oxide (NO) receptor central to NO signaling, which regulates vascular tone in response to endogenously produced NO as well as platelet activation, wound healing and other factors of importance to cardiovascular health. The NO signaling is compromised in numerous forms of vascular pathology and the components of NO signaling pathways are highly sought after therapeutic targets.

sGC is targeted pharmaceutically to treat numerous vascular disorders, including acute coronary syndromes, congestive heart failure, and arterial hypertension, through the use of NO-donors and organic nitrates. While these compounds exhibit potent vasodilatory and anti-ischemic effects, tolerance readily develops and cellular damage by excess NO can occur. More recently, compounds that increase cGMP production without altering cellular levels of NO have been sought. sGC stimulators were the first compounds to overcome the limitations of NO-donors and organic nitrates by enhancing cyclase activity both independently and synergistically with NO. Optimization of initial stimulator compounds led to the development of BAY 41-2272, which is widely used for investigating stimulator mechanism, and BAY 63-2521 (riociguat), which is clinically prescribed to treat pulmonary hypertension. New sGC stimulators are in development, including IWP-051, a novel compound representing a new class of stimulators with improved solubility over traditional stimulators and favorable pharmacodynamics properties.

Despite much success and compounds in the clinic, where sGC stimulators bind and how they function remains unknown. Hence, in one embodiment, the present invention features a method which identifies binding of stimulator compounds to the sGC heme domain and bacterial H-NOX homologs using a unique photoactive cross-linking stimulator with a signature cleavage pattern that allows for unambiguous LC-MS/MS peptide assignment. Various embodiments of the method could also allow for the characterization of a wide variety of important binding sites.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

The present invention describes a method of identifying a crosslinking site on a protein. In some embodiments, the protein may comprise a binding site in a vicinity of the crosslinking site and identification of the crosslinking site may aid in identifying a location of the binding site in the protein. In further embodiments, the method comprises contacting the binding protein with the cross-linking stimulator while exposing them to UV irradiation for a period of time and forming a cross-linked peptide comprising the binding protein and the cross-linking stimulator. In some embodiments, the cross-linked peptide is fragmented to obtain a plurality of identifiable fragments.

In another embodiment, the present invention features a method of identifying a binding site on a protein. In still another embodiment, the present invention may feature a method of identifying a peptide or protein from a complex mixture. In a non-limiting example, the present invention is used to analyze the binding site of sGC, show that stimulator compounds bind to the H-NOX domain of sGC and show that binding also occurs in bacterial homologs.

According to some embodiments, the binding site of said binding protein with the cross-linking stimulator was characterized. In some embodiments, the cross-linking stimulator comprises a photo-cleavable diazirine moiety which transforms into a reactive carbene radical upon UV irradiation and the carbene radical readily reacts with the binding protein to form the cross-linked peptide.

In some embodiments, the plurality of identifiable fragments was characterized through a tandem mass spectrometry (MS₂) with collision-induced dissociation (CID) of the doubly-charged (2+) precursor. In other embodiments, the plurality of identifiable fragments comprises a singly charged fragment ion with m/z 270.127 comprising a biotin unit of the cross-linking stimulator with an amide group, and an ion comprising the cross-linked peptide mass plus the stimulator mass (minus nitrogen).

One of the unique and inventive technical features of the present invention is the use of a crosslinking agent which both comprises a substituent configured to fragment in tandem mass spectrometry to yield a signature mass fragment and is configured to interact with a binding site of a protein. Without wishing to limit the invention to any theory or mechanism, it is believed that the technical feature of the present invention advantageously provides for identification of said binding site. None of the presently known prior references or work has the unique inventive technical feature of the present invention.

### BRIEF DESCRIPTION OF THE DRAWINGS

FIG. 1A shows a flow chart for a method of identifying a crosslinking site on a protein.

FIG. 1B shows a flow chart for a method of identifying a binding site on a protein.

FIG. 10 shows a flow chart for a method of identifying a peptide or protein in a complex mixture.

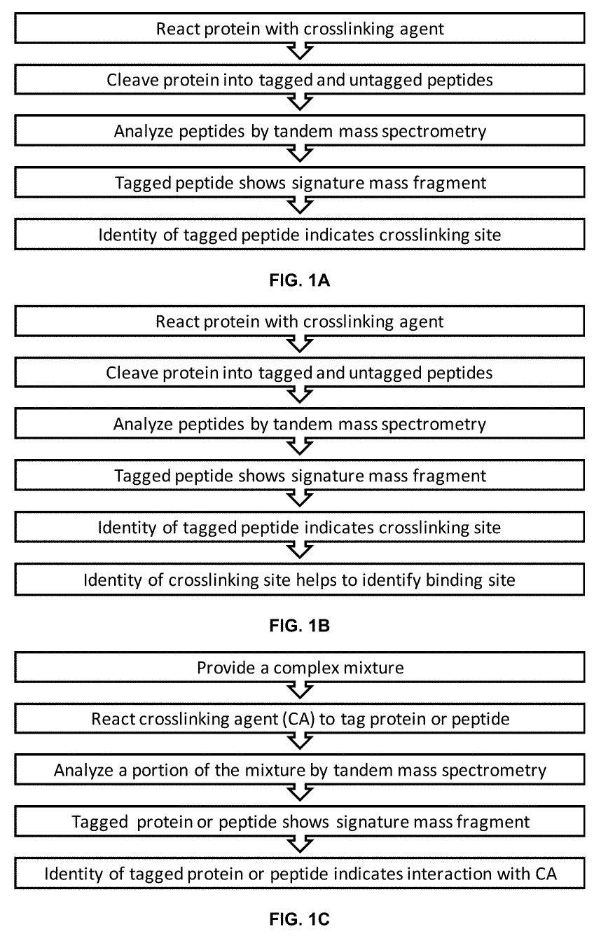

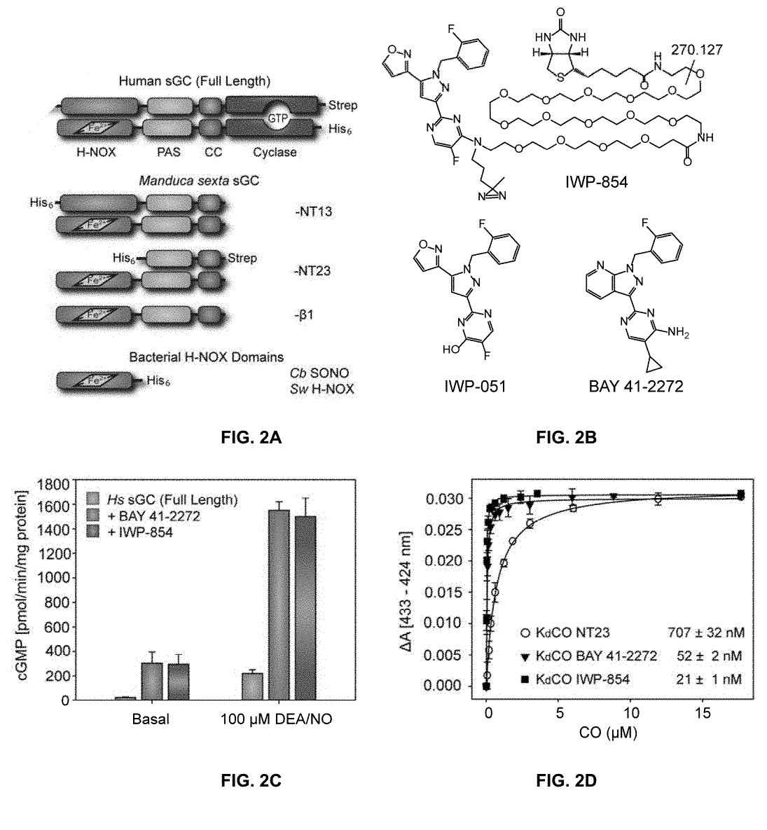

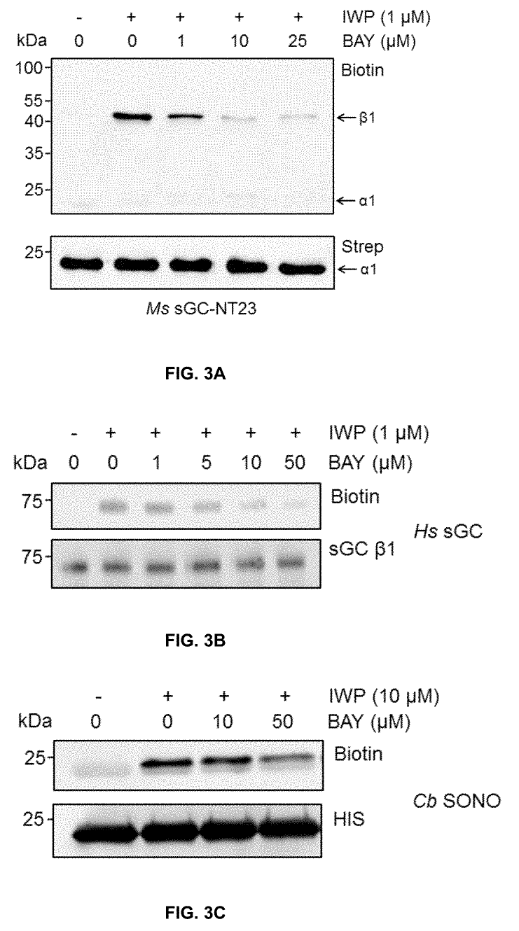

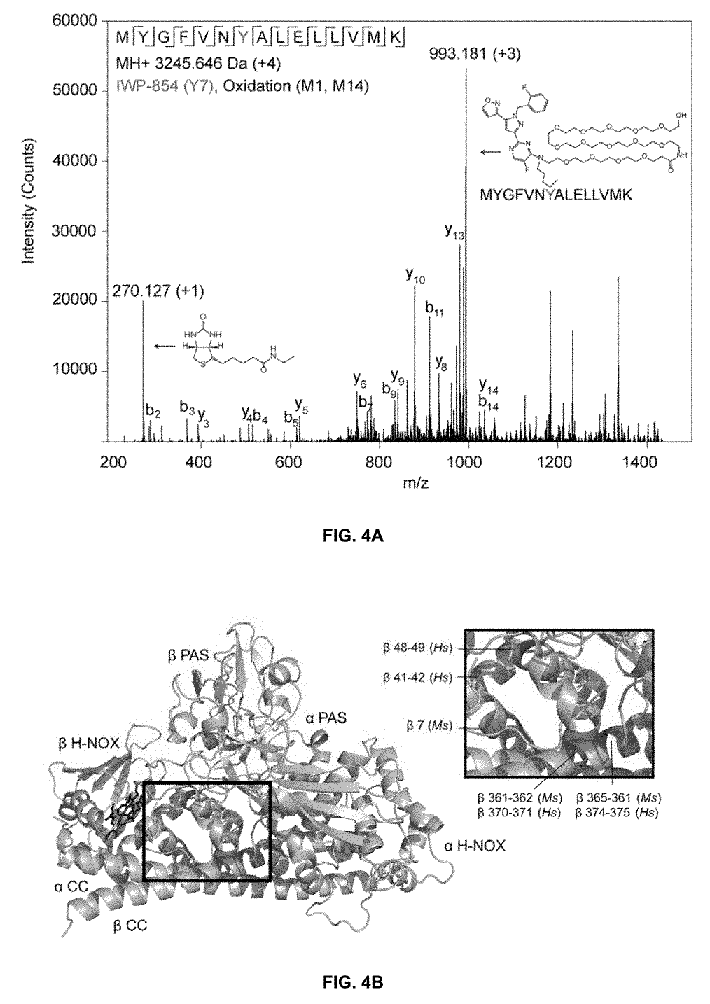

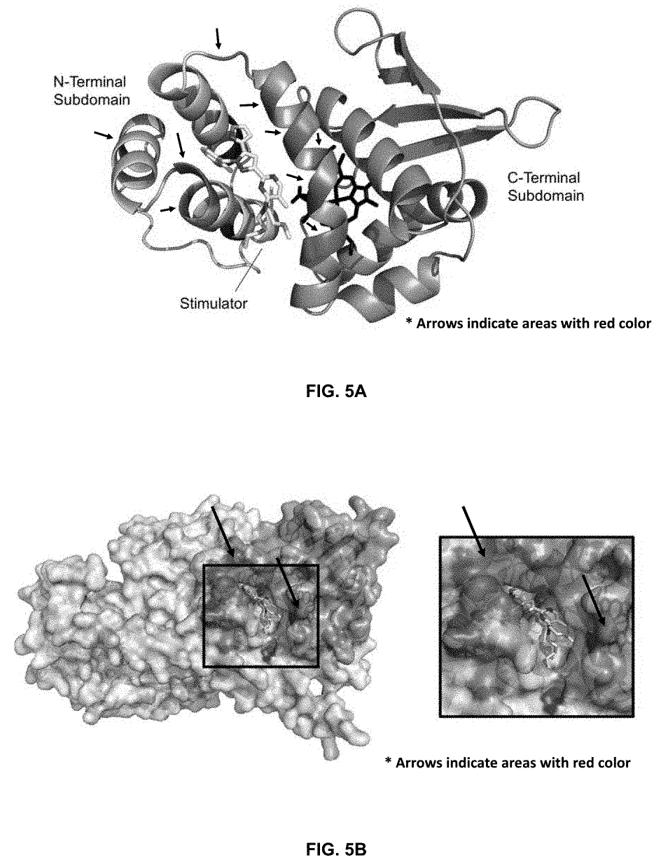

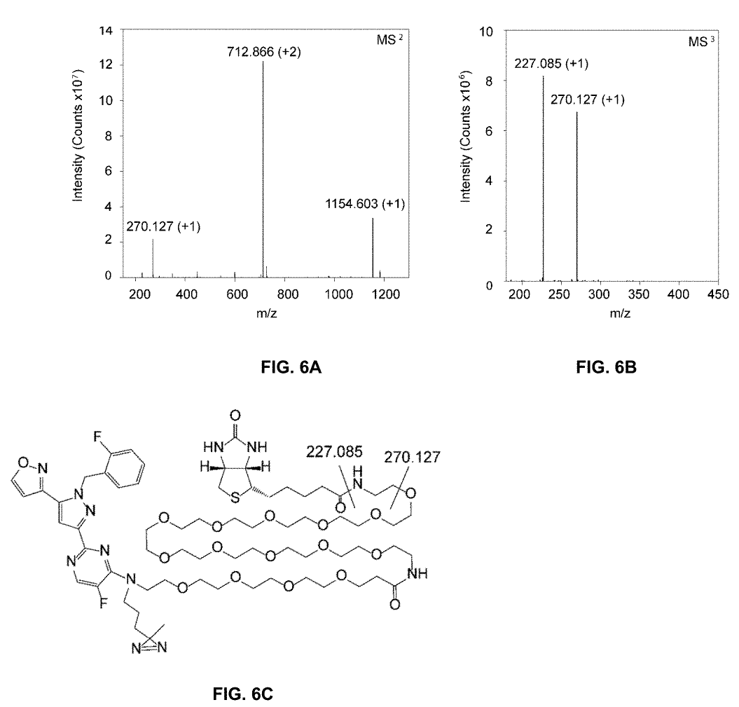

FIG. 2A shows diagram highlighting the domain structure of sGC constructs included in the present example. In addition to full-length human sGC, several truncated versions of Manduca sGC were examined, including Ms sGC-NT13 (α1 49-450, (β1 1-380), Ms sGC-NT23 (α1 49-459, (β1 1-389) and Ms sGC-β1 (β1 1-380), which may be homodimeric. Bacterial H-NOX proteins from Clostridium botulinum (Cb SONO 1-186) and Shewanella woodyi (Sw H-NOX, 1-182) were also examined.

FIG. 2B shows Chemical structures for IWP-854, IWP-051 and BAY 41-2272. The fragmentation position for IWP-854 during mass spectrometry is indicated (270.127).

FIG. 2C shows catalytic activity for human sGC in the absence or presence of DEA/NO (100 μM) and stimulator (5 μM). sGC α1 and β1 genes were transiently transfected into HEK293T cells and activity measured after cell lysis. Error bars represent the average and standard deviation for three independent measurements.

FIG. 2D shows saturation curves for CO binding to Ms sGC-NT23 in the absence or presence of stimulator (5 μM). Error bars represent the average and standard deviation for three independent measurements.

FIG. 3A shows representative western blot illustrating IWP-854 crosslinking to Ms sGC-NT23. Selective cross-linking of IWP-854 (1 μM) to the β1 subunit Ms sGC-NT23 (1 μM) is readily visualized with an anti-biotin antibody after 15 minutes of irradiation with 350-365 UV light (β1 MW=˜45 kDa). In contrast, cross-linking to α1 is not observed (α1 MW=˜20 kDa). Addition of BAY 41-2272 (1-25 μM) competes away IWP-854 in a concentration-dependent manner without increasing non-specific cross-linking. In the lower panel, the Strep-affinity tag at the C-terminus of Ms sGC-NT23 α1 is probed as a loading control.

FIG. 3B shows using full-length recombinant human (Hs) sGC, IWP-854 (1 μM), and BAY 41-2272 (1-50 μM). In the lower panel, Hs sGC β1 is probed as a loading control.

FIG. 3C shows similar to panel (a) using Cb SONO (10 μM, MW˜23 kDa), IWP-854 (10 μM), and BAY 41-2272 (10-50 μM). Cb SONO was reduced to the ferrous state (Soret 431 nm) with 2 mM dithionite and saturated with CO (100 μM). Dithionite was removed from the sample using desalting spin-columns prior to stimulator addition. In the lower panel, the His₆-affinity tag on the C-terminus of Cb SONO is probed as a loading control.

FIG. 4 shows a Mass spectrum of an Ms sGC-NT23 peptide modified by IWP-854. LC-MS/MS spectrum of peptide β1 1-15 modified by IWP-854, undertaken in high resolution/high resolution mode. Loss of the protonated biotin-containing fragment is clearly indicated (m/z=270.127) along with the z=+3 cross-linked peptide (m/z=993.181). Chemical structures for the biotin-containing fragment (m/z=270.127) and the target peptide modified by IWP-854 (m/z=993.181) are depicted in the figure. Nearly all possible b and y ions were observed. Cross-linking was to Tyr 7.

FIG. 5A shows model for the β1 sGC H-NOX domain with bound cross-linking compound. The N-terminal sub-domain and the C-terminal subdomain are labeled. Prominent cross-linked positions are numbered and shown in red (marked by arrows). IWP-854 binding is proposed to be in the pocket formed between sub-domains. One such possibility is shown (sticks; peg linker and biotin left out for clarity). Heme is shown as a stick model.

FIG. 5B shows approximate domain arrangement proposed for Ms sGC-NT13 based on SAXS and chemical cross-linking. Colors for the β1 H-NOX are the same as in panel (a). Cross-links to β1 residues 361 and 366 (coiled-coil) are highlighted in red (marked with arrows).

FIG. 6A shows Mass spectrometry data which was undertaken on IWP-854 without exposure to UV light. MS² of IWP-854 (MW 1,450.743 Da) revealed three prominent peaks: m/z 712.866 (+2), which represents the loss of N₂ (MW 28.013 Da); m/z 1154.603 (+1), which represents the loss of N₂ and fragment 270.127; and the fragment with m/z 270.127 (+1).

FIG. 6B shows Mass spectrometry data which was undertaken on IWP-854 without exposure to UV light. MS³ of fragment m/z 270.127 which revealed a fragment at m/z 227.085, and allowed the m/z 270.127 to be identified as the end of the biotin linker.

FIG. 6C shows the structure of IWP-854 with the m/z 270.127 and m/z 227.085 fragments indicated.

### DESCRIPTION OF PREFERRED EMBODIMENTS

In one embodiment, the present invention features a method of identifying a crosslinking site on a protein. As a non-limiting example, the method may comprise: reacting said protein with a crosslinking agent at the crosslinking site to form a crosslinked protein, wherein the crosslinking agent comprises a substituent configured to fragment in tandem mass spectrometry to yield a signature mass fragment; cleaving said protein into two or more peptides, wherein at least one of the peptides is tagged by the crosslinking agent; and analyzing the peptides by tandem mass spectrometry to detect the tagged peptide, wherein said tagged peptide fragments to yield the signature mass fragment, wherein the signature mass fragment is detected to identify the tagged peptide and wherein identification of the tagged peptide indicates the crosslinking site on the protein. In some embodiments, the protein may comprise a binding site in a vicinity of the crosslinking site and identification of the crosslinking site may aid in identifying a location of the binding site in the protein.

In another embodiment, the present invention features a method of identifying a binding site on a protein. As a non-limiting example, the method may comprise: reacting said protein with a crosslinking agent at a crosslinking site in a vicinity of the binding site, to form a crosslinked protein, wherein the crosslinking agent comprises a substituent configured to fragment in tandem mass spectrometry to yield a signature mass fragment; cleaving said protein into two or more peptides, wherein at least one of the peptides is tagged by the crosslinking agent; and analyzing the peptides by tandem mass spectrometry to detect the tagged peptide, wherein said tagged peptide fragments to yield the signature mass fragment, and wherein the signature mass fragment is detected to identify the tagged peptide; wherein identification of the tagged peptide indicates the crosslinking site on the protein and wherein identification of the crosslinking site aids in identifying a location of the binding site in the protein.

In still another embodiment, the present invention may feature a method of identifying a peptide or protein from a complex mixture. As a non-limiting example, the method may comprise providing the complex mixture; introducing a crosslinking agent which selectively interacts with the peptide or protein, and reacts with the peptide or protein to form a tagged peptide or protein, wherein the crosslinking agent comprises a substituent configured to fragment in tandem mass spectrometry to yield a signature mass fragment; and analyzing a portion of the complex mixture by tandem mass spectrometry to detect the tagged peptide or protein, wherein said tagged peptide or protein fragments to yield the signature mass fragment, and wherein the signature mass fragment is detected to identify the tagged peptide or protein. In some embodiments, the complex mixture may comprise at least one of the following: a peptide, protein, biomolecule, biopolymer, cell, cell lysate, pharmaceutical agent, DNA strand, organelle, or small-molecule.

In some embodiments, the crosslinking agent may be configured to interact with said binding site. As a non-limiting example, the binding site may be a receptor and the crosslinking agent may comprise an agonist or antagonist of said receptor. In another embodiment, the binding site may be a drug binding site and the crosslinking agent may comprise a modified drug. In yet another embodiment, the protein may comprise a guanylyl or guanylate cyclase, a truncated version of guanylyl cyclase or a bacterial H-NOX homolog.

According to one embodiment, the crosslinking agent may comprise a photo-cleavable diazirine moiety which transforms into a reactive carbene radical upon UV irradiation and the carbene radical reacts with the protein to form the crosslinked or tagged protein or peptide. In preferred embodiments, the duration of UV irradiation of the crosslinking agent may be about 5 min. In other embodiments, the duration of UV irradiation of the crosslinking agent may be about 0.5, 1, 2, 3, 7, 10, 15, 20 or 30 min.

According to some preferred embodiments, a bond linking an amide functional group and an ether functional group may be broken during the analysis of the tagged peptide or protein, thereby forming the signature mass fragment having an m/z ratio value. Without wishing to limit the invention to any theory or mechanism, it may be that the nitrogen or the amide is protonated and this protonation aids in the breaking of the bond linking the amide group to the ether group. In other embodiments, the m/z ratio value of the signature mass fragment may correspond to an ion comprising the tagged peptide or protein mass. In still another embodiment, the signature mass fragment may correspond to a singly charged fragment ion which comprises a biotin moiety with an amide substituent and has a m/z ratio of 270.127. As a non-limiting example, the substituent configured to fragment may be according to the following structure:

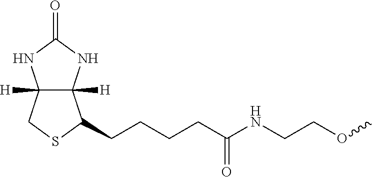

According to one embodiment, the peptides or proteins may be dissolved in acidic solvent before being analyzed by tandem mass spectrometry. In another embodiment, the crosslinking agent is according to the following structure:

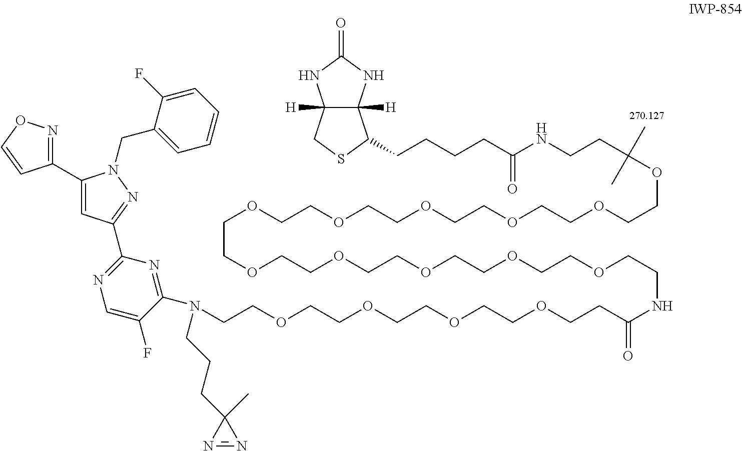

The following is provided as a non-limiting example of the invention. Equivalents or substitutes are within the scope of the invention

Use of a Photolyzable Stimulator, IWP-854 (Developed by Ironwood Pharmaceuticals).

To localize stimulator binding to sGC, a photolabile compound called IWP-854 was utilized. The IWP-854 core motif is based on IWP-051, which replaces the fused-ring system with two five-membered rings capable of free rotation (FIG. 2). The pyrimidine ring was modified to have a biotin affinity tag coupled to a PEG linker and a photoactive diazirine capable of cross-linking to all 20 amino acid side chains and peptide backbone (FIG. 2).

Diazirines generate a highly reactive carbene in response to irradiation with 350-365 nm UV light, which rapidly inserts into neighboring C—C, C—H, O—H, and N—H bonds. Robust cross-linking is seen to all 20 amino acid side chains as well as to peptide backbone atoms. These properties have made diazirines popular in photoaffinity labeling experiments seeking drug/protein binding sites and are ideal for localizing stimulator binding to sGC.

IWP-854 Retains Stimulator Activity.

Stimulation of recombinant human (Hs) sGC was examined and IWP-854 was found to stimulate to a similar extent as BAY 41-2272 (FIG. 2). Both basal and NO stimulated activities were enhanced. IWP-854 increased activity by 12-fold over basal levels in the absence of added NO, and 84-fold over basal upon addition of NO.

A truncated version of sGC from Manduca sexta was developed for analyses of compound-enhanced CO binding. Herein, these constructs are referred to as Ms SGC-NT. Ms sGC-NT constructs are fully heme-loaded and stable in the ferrous (functional) state, as indicated by Soret band absorption. One hallmark of stimulator compounds is their ability to enhance CO and NO binding to the heme domain. CO binding to Ms sGC-NT23 in the absence of stimulator compound displays KdCO=710 nM (FIG. 2). Addition of BAY 41-2272 enhances CO affinity 14-fold (KdCO=52 nM) while addition of IWP-854 enhances CO affinity 34-fold (KdCO=21 nM). Thus, IWP-854 stimulates as well or somewhat better than the best previously described compounds.

IWP-854 and BAY 41-2272 Share a Common Binding Site.

Cross-linking with IWP-854 was visualized by probing the biotin affinity tag through western blot (FIG. 3). A time course revealed that cross-linking to Ms sGC-NT23 is observed after 5 minutes of UV illumination and continues to increase for 15-20 minutes. Heme was retained after 15 minutes of UV irradiation, as indicated by a minimal decrease in Soret band absorption and a slight shift in Soret maxima characteristic of stimulator binding. Longer exposures to UV light led to substantial heme loss, however, and were avoided for this reason. Using this strategy, it was found that IWP-854 cross-links exclusively to the β1 subunit of Ms SGC-NT23 (FIG. 3A).

To assess compound specificity, IWP-854 cross-linking was monitored in the presence of increasing concentrations of BAY 41-2272 (FIG. 3A). Incubation with BAY 41-2272 attenuated IWP-854 cross-linking to Ms sGC-NT23 in a concentration-dependent manner, indicating the two compounds were competing for a single binding pocket. Similar results were observed for Ms SGC-NT13, which includes the α1 pseudo H-NOX domain, and full-length Hs SGC (FIG. 3B). Faint labeling was observed on the α1 subunit of Hs sGC; however, this labeling was not competed away with excess stimulator and is likely due to non-specific cross-linking. IWP-854 also labeled the isolated β1 subunit of Ms sGC-NT21 (β31 residues 1-380). Curiously, BAY 41-2272 failed to diminish IWP-854 labeling of Ms sGC-β1; however, IWP-854 labeled the same residues in Ms sGC-β1 as other Ms sGC-NT constructs (Table 1).

Stimulator binding to Cb SONO was characterized, as well as three H-NOX proteins from Nostoc sp. 7120 (Ns H-NOX), Shewanella oneidensis (So H-NOX), and Shewanella woodyi (Sw H-NOX). IWP-854 labeled all four bacterial H-NOX proteins (FIG. 3C), suggesting stimulator binding is conserved among β1 H-NOX domains. Photoaffinity labeling (PAL) of bacterial H-NOX proteins required 10-fold more IWP-854 than SGC constructs, indicating decreased affinity for compound binding. Labeling was reduced but not eliminated by excess BAY 41-2272, which is likely explained by the inability to reach sufficiently high BAY 41-2272 concentrations due to poor compound solubility, and by the increased nonspecific cross-linking that occurs at higher IWP-854 concentrations. Interestingly, BAY 41-2272 did not enhance CO binding to any of the four bacterial H-NOX proteins.

Identifying Cross-Linked Residues in sGC and Bacterial H-NOX Proteins.

Residues labeled by IWP-854 were identified by liquid chromatography tandem mass spectrometry (LC-MS/MS) using an LTQ Velos Orbitrap mass spectrometer. Initial examination of IWP-854 alone (molecular mass 1,450.743 Da) revealed a distinct and highly reproducible fragmentation pattern (FIG. 6A). Key features include a singly-charged peak at m/z 270.127 and a peak one charge state less than the precursor representing the mass of the parent ion minus 270.127 Da. MS analysis of m/z 270.127 identified the fragment as the end of the biotin-containing PEG linker (C12H20O2N3S, FIG. 2B and FIG. 6A). This signature cleavage pattern was consistently observed in cross-linked peptides (FIG. 4), providing a robust strategy for definitively identifying peptides labeled by IWP-854. A possible mechanism for the characteristic fragmentation pattern is for the linker amide near the cleavage site to yield a localized mobile proton that assists in the cleavage event, thus reproducibly generating the m/z 270.127 fragment. More broadly, judicious placement of an amide bond in a PEG linker may provide a unique cleavage pattern of general applicability.

The availability of multiple Ms sGC-NT constructs in high purity and abundance allowed for numerous experiments to be undertaken under varying conditions. Hs sGC and four bacterial H-NOX proteins were also examined using similar conditions to those initially developed with Ms sGC-NT. Results from a total of 43 experiments are reported in Table 1 and Table 2.

Identification of peptides was to high mass accuracy in all cases; however, certain peptides were detected more often than others (Table 1). The majority of labeled residues identified in Ms sGC-NT and Hs sGC are expected to originate from the stimulator-binding pocket, as evidenced by diminished cross-linking in the presence of excess BAY 41-2272 (FIG. 3). In general, the diversity in labeling of sGC likely results from dynamics in both compound and protein. For IWP-854, the diazirine is at the fourth carbon of a 5-atom flexible linker and is further attached to a pyrimidine ring capable of free rotation (FIG. 2B). The pyrimidine ring is highly tolerant of modifications, suggesting it does not make critical contacts with the protein and may exhibit greater conformational dynamics than the rest of the compound, even in the bound state.

Labeling of Ms sGC-NT23 remained the same ±NO or CO, consistent with a stimulator binding site that does not greatly change upon heme ligation. Likewise, labeling did not appreciably differ in the presence (Ms sGC-NT13) or absence (Ms sGC-NT23) of the α1 pseudo H-NOX domain, indicating this domain does not harbor the stimulator binding site. IWP-854 labeling of Ms sGC-β1 is similar to the other Ms sGC-NT constructs despite lacking the α1 chain and displaying poor competition with BAY 41-2272. Labeled peptides identified in the Ms sGC-NT constructs agreed well with those in full-length Hs sGC and were found nearly exclusively in the β1 subunit, as expected from the western blot analyses. Likewise, many labeled peptides identified in the bacterial H-NOX proteins overlapped with those from sGC.

The most frequently observed cross-links cluster together in previous models for Ms sGC-NT. IWP-854 predominately labeled the sGC β1 chain on H-NOX alpha helix αA (Ms residues β1 6-8), H-NOX helix αC (Hs residues β1 48-51), and the coiled-coil domain (Ms residues β1 361-362, 365-366; Hs residues β1 370-371, 374-375, 385). Cross-linking to helix αA was also seen for the bacterial H-NOX proteins Cb SONO and Sw H-NOX, while cross-linking to helix αC was seen for Ns H-NOX, So H-NOX, and Sw H-NOX. Helix αD was not labeled in any sGC constructs but was detected in bacterial H-NOX proteins Ns H-NOX, Sw H-NOX, and Cb SONO. Labeled residues in αA, αC, and αD localize around the interface of two subdomains in the β1 H-NOX and are predicted to reside near cross-linked residues in the coiled-coil and the linker connecting the PAS and coiled-coil domains of Ms sGC-NT (Ms residues β1 328-331).

Cross-links to residues Ms sGC-NT β1 195-198 were seen in 11 of 26 measurements; however, these residues lie in the linker between the H-NOX and PAS domains and in a different region of the model. This discrepancy could be due to limitations in modeling, a slight degree of non-specific binding or high dynamics in this loop.

A number of additional cross-linked peptides were detected on a less frequent basis in these experiments, and are listed in Table 1 and Table 2. Modifications to the β1 H-NOX/PAS linker were observed in all three Ms sGC-NT constructs, but not Hs sGC. Additionally, a variety of cross-links were detected in individual bacterial H-NOX proteins that do not agree with the most common binding arrangement. These are likely the result of unspecific labeling introduced by increased cross-linker concentrations and/or changes in compound affinity, as evidenced by incomplete elimination of IWP-854 cross-linking by competition with BAY 41-2272. For this reason, only labeled residues that were identified in multiple bacterial H-NOX proteins were considered to be part of the binding site.

Stimulators have been proposed to bind to a pseudosymmetric site in the cyclase domains similar to forskolin binding to adenylyl cyclase. Ms sGC-NT constructs retain stimulator binding and response despite lacking both cyclase domains, suggesting the primary stimulator binding site resides in the N-terminal two-thirds of the protein. The possibility of a secondary stimulator binding site in the catalytic domains was examined using photoaffinity cross-linking of full length Hs sGC. A single cross-link was found in cyclase domain (Hs residue α1 629, Table 2), which lies on the surface of the protein near where the coiled-coil attaches. No cross-links were found to residues in the cyclase domain active site or pseudosymmetric site, rendering the possibility of a secondary stimulator binding site unlikely. One additional cross-link to the human α1 chain was observed (Hs peptide α1 45-47, Table 2). The two α1 chain cross-links may represent the non-specific α1 cross-link found by western blotting.

Molecular Modeling of Stimulator Binding to sGC.

The cross-linking data indicate the primary binding site for stimulator compounds is in the β1 H-NOX domain. While a high-resolution structure of an sGC H-NOX domain has not been reported, crystal structures for several bacterial homologues are known, including those from Thermoanaerobacter tengcongensis (Tt H-NOX, recently renamed as Caldanaerobacter subterraneus, Cs H-NOX), Nostoc sp. (Ns H-NOX and Shewanella oneidensis (So H-NOX). These structures display the same overall fold and provide a solid scaffold for understanding H-NOX structure in sGC function. The overall H-NOX fold is ˜180 residues long and displays an N-terminal sub-domain encompassing residues 1-60, which is dominated by a 3-helix bundle, followed by a larger mixed helix/sheet sub-domain that contains the heme pocket. Alignment of the larger sub-domains of several H-NOX structures indicate the smaller and larger domains can move independently of one another, altering the orientation of the two domains, which may be key for signal transduction by H-NOX domains and proteins.

Most of the residues implicated in compound binding by cross-linking lie at the interface of the small and large H-NOX domains (FIG. 5A), suggesting this interface provides the binding pocket. While the large domain includes the heme-binding pocket, the small domain covers the heme distal pocket and contacts the heme edge. Changes in the sub-domain interface may possible affect heme properties, ligand affinity and signal transduction. Thus, the working hypothesis is that stimulator compounds bind in this region and IWP-854 binding has been modeled into a pocket at the sub-domain interface (FIG. 5A).

Modeling of compound into heterodimeric sGC was more challenging since atomic-level models are unavailable. For this, a model for Ms sGC-NT based on small-angle X-ray scattering (SAXS) was utilized, chemical cross-linking and homology modeling of the H-NOX, PAS and coiled-coil domains. The cross-links found most frequently in the present example were to the coiled-coil. Encouragingly, these residues residue near the labeled residues in the αA, αC, and αD helixes of β1 H-NOX domain (FIG. 5B).

Discussion

In the present example, a photoactivatable stimulator compound coupled with LC-MS/MS was used to narrow the binding site to the β₁ H-NOX domain. No binding to the cyclase domain active site or pseudosymmetric site was observed, nor was there binding to the α1 H-NOX domain. The discrepancy likely results from choice of cross-linker, with the present example utilizing a diazirine versus an aryl azide in the former. Diazirines improve upon aryl azides with quicker reaction times and a lower frequency of stable intermediates that are capable of diffusing away from the binding site, which in the case of aryl azides, includes ketenimine decay products that react strongly with nucleophiles such as cysteines. Since both residues previously identified were cysteines, the reaction may have been with the ketenimine.

A possible binding complex in which stimulators bind at the interface of the two H-NOX subdomains was modeled, where the majority of the cross-links were located (FIG. 5A). This pocket is part of a tunnel suggested to be of importance for NO, CO and O₂ gas exchange with the heme distal pocket in bacterial H-NOX proteins. Filling this pocket with compound provides a possible mechanism for stimulation and may explain the conservation of binding in H-NOX proteins. With the most critical portions of stimulator compounds filling the gas-exchange tunnel, the IWP-854 pyrimidine ring, which contains the diazirine cross-linker, was modeled to be near the major H-NOX and coiled-coil cross-linked peptides (FIG. 5B). Since the coiled-coil was labeled in all experiments in which it was present, these results suggest a compact domain arrangement of sGC is likely to occur in high abundance.

In summary, the present example has shown that stimulator compounds bind to the H-NOX domain of sGC and that binding also occurs in bacterial homologs. Binding likely occurs at the interface of the H-NOX large and small sub-domains, and may act through both inducing an active conformation and through directly blocking a tunnel for gas release to bulk solvent. These data provide insight into sGC function and stimulator action, and provide a roadmap for improved compounds targeting cardiovascular disease. A photolyzable cross-linking compound with a signature cleavage pattern that may be of general applicability has also been described.

### Methods

### Example 1: The Following are Exemplary Synthetic Procedures, and are Included Herein as Non-Limiting Example

Syntheses.

IWP-051 and IWP-898 (identical to phosphodiesterase 9A inhibitor PF-044447943) were produced. Photoactive cross-linker IWP-854 was synthesized and purified in a manner similar to IWP-051.

Materials:

Chemicals were purchased from Sigma Aldrich unless otherwise indicated. Uniformly labeled 15N-ammonium chloride and deuterium oxide (D2O) were purchased from Cambridge Isotope Laboratories. 2-(N,N-Diethylamino)diazenolate-2-oxide (DEA/NO) was provided by Dr. Katrina Miranda from the University of Arizona. Full-length human sGC was purchased from Enzo Life Sciences, Inc. HEK293T cells were acquired from the American Type Culture Collection (ATCC). Turbofect was purchased from Fermentas. DMEM media was purchased from Gibco Life Technologies. Fetal bovine serum was obtained from the University of Arizona Cancer Center. cGMP was measured using a commercially available homogenous time-resolved fluorescence (HTRF) immunoassay (CisBio). Sequencing grade trypsin was acquired from Promega, and C18 zip-tips were purchased from Pierce Thermo Fisher Scientific.

Generation of Ms sGC-NT Constructs.

Multiple N-terminal truncations of Manduca sexta sGC (Ms sGC-NT) were utilized in the present example (FIG. 2A, 2B). Construction of Ms sGC-NT13 (α1 His₆-49-450, β1 1-380) and Ms sGC-NT21 (α1 His₆-272-450, β1 1-380) was previously described. Two novel constructs were generated in the present work, Ms sGC-NT23 (α1 His₆-272-459-Strep, β1 1-389) and Ms sGC-NT25 (α1 His₆-49-459-Strep, β1 1-389). Ms sGC-NT23 and Ms sGC-NT25 resemble Ms sGC-NT21 and Ms sGC-NT13, respectively, with the exception that the C-terminus of the α1 and β1 subunits were elongated by 9 residues and the al subunit contains a C-terminal Strep purification tag (WSHPQFEK).

cDNA coding for Ms sGC residues α₁ 49-459 was PCR amplified from plasmid pETDuet-1-msGC-NT2 using primers F1 and R1. The PCR product was subcloned into vector pGEM-T. After digestion with BamHI and NotI, the new α₁ subunit was cloned into plasmid pETDuet-1-NT2 following removal of the original α₁ subunit using the same restriction enzymes. The Ms sGC β₁ subunit was shortened by inserting a stop codon (TAA) after Leu 389 using the QuikChange Lightning Site-Derected Mutagensis Kit with primers F2 and R2. The resulting pETDuet-1-NT25 plasmid codes for Ms sGC-NT25.

Ms sGC-NT23 was generated from Ms sGC-NT25 by removing the α₁ pseudo H-NOX coding sequence (α₁ 49-271). To accomplish this, an equimolar mixture of DNA oligonucleotides F3 and R3 were annealed by heating at 95° C. for 2 min and cooling to room temperature, yielding a 37-base double-stranded DNA with 4-base overhangs on each end. This was ligated into the pETDuet-1-NT25 plasmid with T4 ligase after removal of the α₁ H-NOX domain with BamHI and NheI restriction enzymes. The resulting plasmid, pETDuet-1-NT23, codes for Ms sGC-NT23.

Expression and Purification of Ms sGC-NT.

Ms sGC-NT13 and Ms sGC-NT21 were expressed and purified from Eschercheria coli. Ms sGC-β (β1 1-380) was purified by washing Ms sGC-NT21 bound to a TALON Superflow Metal Affinity column with 50 mM sodium phosphate buffer (pH 7.4), 300 mM NaCl saturated with 1 mM CO. CO binding to Ms sGC-NT21 led to selective elution of the β1 subunit while the al subunit remained bound to the column.

Ms sGC-NT23 was expressed in the same manner as Ms sGC-NT13 and Ms sGC-NT21. For purification of Ms sGC-NT23, pellets were resuspended in 50 mM sodium phosphate buffer (pH 7.4), 300 mM NaCl, 0.75 mM DNase I, and protease inhibitors (2 mM MgCl2, 1 mM PMSF, 1 μg/mL aprotinin, 1 μg/mL leupeptin, 0.25 mg/mL lysozyme, 1 mM benzamine), and lysed by French press. Cell debris was removed by ultracentrifugation at 40,000 rpm in a Ti45 rotor for 35 min at 4° C. The sample was loaded onto a TALON Superflow Metal Affinity column and washed with 50 mM sodium phosphate (pH 7.4), 300 mM NaCl. Protein was eluted by supplementing wash buffer with 30 mM EDTA. Protein fractions were loaded onto a Strep-tactin Sepharose column and eluted with 3 mM desthiobiotin in 50 mM sodium phosphate buffer (pH 7.4), 100 mM NaCl, 5% glycerol. Samples were concentrated using Viva spin 30 kDa protein concentrators and flash frozen in liquid nitrogen for storage at −80° C.

Expression and Purification of Bacterial H-NOX Proteins.

The genes coding for H-NOX proteins from Nostoc sp. PCC 7120 (Ns H-NOX; residues 1-182; NCBI Ref Seq: WP_010996435.1), Shewanella oneidensis (So H-NOX; residues 1-181; NCBI Ref Seq: WP_011072197.1), Shewanella woodyi (Sw H-NOX; residues 1-182; NCBI Ref Seq: WP_012325363.1), and Clostridium botulinum (Cb SONO; residues 1-186; NCBI Ref Seq: WP_012048396.1) were synthesized and cloned into the pET21b+ vector between restriction sites NdeI and XhoI by GenScript Biotech Corporation. All bacterial H-NOX proteins were engineered to contain a C-terminal TEV cleavage sequence and a 6× poly-histidine purification tag (ENLYFQSLEHHHHHH).

With the exception of Sw H-NOX, the plasmid coding for the bacterial H-NOX protein of interest was transformed into Rosetta competent cells and grown to an OD₆₀₀ of 0.8-1.0 in Terrific Broth media at 37° C. while shaking at 225 rpm. Protein expression was initiated by adding 0.5 mM IPTG and 0.1 mM δ-aminolevulinic acid, and continued for 18-22 hours at 16° C. The plasmid coding for Sw H-NOX was transformed into Escherichia coli Tuner (DE3) pLysS competent cells and grown to an OD₆₀₀ of 0.8-1.0 in M9 media at 37° C. Protein expression was induced with 0.1 mM IPTG and 0.05 mM δ-aminolevulinic acid, and expressed for 18-22 hours at 22° C. All bacterial cultures were harvested in the same manner as Ms sGC-NT constructs.

Pellets containing were resuspended in 50 mM sodium phosphate buffer (pH 7.4), 300 mM NaCl, 0.75 mM DNase I, 1 mM PMSF and lysed by French press. Cell debris was removed by ultracentrifugation at 40,000 rpm in a Ti45 rotor for 35 min at 4° C. The supernatant was loaded onto a Ni-NTA affinity column and washed with 50 mM sodium phosphate (pH 7.4), 300 mM NaCl. Ns H-NOX and So H-NOX were eluted with wash buffer supplemented with 30 mM EDTA. Sw H-NOX and Cb SONO required higher salt concentrations for elution and were eluted in 50 mM sodium phosphate buffer (pH 7.4), 500 mM NaCl, 30 mM EDTA. Buffer was exchanged to 50 mM sodium phosphate (pH 7.4), 90 mM NaCl, <0.1 mM EDTA using Viva spin 10 kDa protein concentrators and the sample was incubated with a 1:100 (protease:protein) ratio of TEV protease (purified in-house) at 4° C. overnight. The reaction solution was passed over a Ni-NTA column and cleaved protein was collected in the flow through. The protein of interest was further purified by gel filtration using Superdex 200 that was pre-equilibrated with 50 mM sodium phosphate buffer (pH 7.4), 100 mM NaCl. Protein concentrations were assessed spectroscopically based on Soret and with the Bradford Protein Assay. All samples were frozen in liquid nitrogen for storage at −80° C.

Ns H-NOX and So H-NOX were purified in the ferrous state (soret maximum 431 nm), while Cb SONO and Sw H-NOX were purified in the ferric state (soret maximum 408 nm and 416 nm, respectively). Cb SONO and Sw H-NOX were fully reduced to the ferrous state (soret maximum 431 nm) immediately prior to use by incubating the sample with 2 mM sodium dithionite in degassed buffer for 10 minutes at room temperature. Sodium dithionite was removed from samples used in PAL experiments using 10 kDa Zebra spin desalting columns.

Determination of CO Dissociation Constants:

A 1 mM CO solution was obtained by bubbling 50 mM sodium phosphate buffer (pH 7.5), 100 mM NaCl, 5% glycerol with CO for 10-20 minutes. The Ms SGC-NT or bacterial H-NOX construct to be analyzed (25 nM) was suspended in 50 mM sodium phosphate buffer, pH 7.5, 100 mM NaCl, 5% glycerol in the presence or absence of 5-10 μM IWP-854 or BAY 41-2272. A final concentration of 5% EtOH was added to the solution to maintain solubility of BAY 41-2272. CO titrations were measured using a Cary350 UV/visible spectrophotometer using a cuvette with 10 cm pathlength. K_{d}^{CO} measurements were calculated.

Generation of Human sGC Constructs:

cDNA sequences for Hs SGC-α (α1 1-690-Strep-tag II) and Hs SGC-β1 (β1 1-619-His₆) were PCR amplified using primers F4, R4, F5 and R5. PCR products for Hs sGC-α1 were cloned into plasmid pCMV_3 TAG9 between restriction sites BAMHI and HindIII and PCR products for Hs SGC-β1 were cloned into pCMV_3 TAG3A between sites SacI and XhoI to create the plasmids pCMV_α1 and pCMV_β1, which code for Hs SGC-α and Hs SGC-β, respectively.

Expression of human sGC.

HEK 293T cells were grown in Dulbecco's Modified Eagle's medium (DMEM) supplemented with 10% fetal bovine serum (FBS) at 37° C. with 5% CO₂. A transfection mixture containing 22.5 μg pCMV_α1, 2.5 μg pCMV_β1, and 31.25 μL Turbofect was assembled in 2.5 mL serum-free DMEM. The transfection mixture was incubated for 30 min at room temperature and added drop wise to a 10 cm dish containing HEK 293T cells grown to ˜75% confluency. Protein expression continued for 24 h at 37° C. with 5% CO₂. Transfection and protein expression were confirmed by western blot, probing for the Strep and His₆ purification tags found on the C-terminus of sGC sGC-α1 and sGC-β1, respectively.

cGMP Measurement.

HEK 293T cells transfected with human sGC were washed twice with PBS and suspended in 50 mM Tris buffer (pH 7.4), 100 mM NaCl, 8 mM MgCl₂, 0.5 mM IMBX, protease inhibitors (1 mM PMSF, 1 μg/mL aprotinin, 1 μg/mL leupeptin, 1:100 dilution of protease inhibitor cocktail) and lysed with 30 stokes of a 25-gauge needle. Cell debris was removed by centrifugation at 16,000 g for 20 minutes at 4° C. Transfected HEK 293T lysate was divided into equal aliquots and combined with 5 μM stimulator (IWP-854 or BAY 41-2272) and/or 100 μM DEA/NO, as indicated. Samples were incubated at room temperature for 10 min to permit NO release and the reaction initiated with 1 mM GTP. Reactions proceeded for 5 min at 37° C. before quenching with 1.5% glacial acetic acid. Precipitated protein was removed by centrifuging at 16,000 g for 10 min at 4° C., and the supernatant was diluted 1:100 in 50 mM sodium phosphate (pH 7.0), 0.2% BSA, 0.2% NaN₃. cGMP was quantified using a commercially available homogenous time resolved fluorescence (HTRF) assay. All samples were measured in duplicate using a SynergyH1 fluorescent plate reader. HTRF measurements were analyzed according to the manufacturer's instructions using Sigma Plot.

Photoaffinity Labeling sGC with IWP-854.

The sGC or bacterial H-NOX construct of interest (1 μM for human sGC or Ms SGC-NT, 10 μM for bacterial H-NOX proteins) was suspended in 50 mM sodium phosphate buffer, pH 7.5, 100 mM NaCl. DEA/NO (100 μM), CO (50 μM), and/or varying concentrations of BAY 41-2272 (1-25 μM for human sGC or Ms SGC-NT, 10-100 μM for bacterial H-NOX proteins) were added. For competition assays, a final concentration of 5% ethanol was added to maintain the solubility of BAY 41-2272. Samples were equilibrated for 10 min at room temperature before adding IWP-854 (1 μM for sGC constructs, 10 μM for bacterial H-NOX proteins). Following a 10 min incubation at room temperature in the dark, samples were placed in a 96-well tissue culture plate and irradiated for 15 minutes on ice with broad band UV light (366 nm maximum) using a multiband UVGL-58 lamp. Protein stability throughout the experiment was assessed based on heme content, which was monitored spectroscopically using a Cary350 UV/visible spectrophotometer.

Western Blot Analysis of Photoaffinity Labeled Samples.

Photoaffinity labeling of IWP-854 to sGC or bacterial H-NOX proteins was visualized by probing the biotin affinity-tag via western blot. Total protein was visualized by probing for Strep or His₆ purification tags, by probing the β1 subunit directly, or by Ponceau stain. With the exception of Hs sGC, all samples were suspended 1×SDS loading buffer and a total of 1 μg protein was run on a 15% bis-acrylamide gel for 90 minutes at 96 V. Samples were transferred to a nitrocellulose membrane at 100 V for 1 h at 4° C. The membrane was blocked for 1 h in 5% BSA dissolved in PBST, and incubated in a 1:1000 dilution of primary antibody (Cell Signal #5597 for probing the biotin affinity-tag of IWP-854; Abcam ab76949 for probing the Strep-tag, QED Biosciences #18814-01 for probing His₆) over night at 4° C. The membrane washed three times in PBST and incubated in secondary antibody (IRDye 680 goat anti-rabbit secondary mAB, Licor 925-68071) for 2 h while shaking at room temperature. Membranes were washed an additional three times and imaged using the Odyssey Infrared Imaging System.

For Hs sGC, samples were suspended in 2× of loading buffer and a total of 200 ng of protein was run on a NuPAGE 4-12% Bis-Tris gel for 60 min at 160 volts. Samples were transferred to a nitrocellulose membrane at 150 volts for 45 min at room temperature. The membrane was blocked for 1 h in PBS with 5% dry fat free milk. The membrane was washed three times with TBS containing 0.1% Tween-20 and incubated for 1 h at room temperature in a 1:2000 dilution of IRdye800 streptavidin, into Odyssey blocking buffer containing 0.2% Tween-20. The membrane was washed three times with TBS-T and imaged using Odyssey CLx imager. Then, as a loading control, membrane was incubated overnight at 4′C in 1:1000 dilution of antibody against sGC β1 subunit into TBS-T with 5% BSA, The membrane was washed three times with TBS-T and incubated for 1 h at room temperature in a 1:15000 dilution of IRdye680 secondary antibody into Odyssey blocking buffer containing 0.2% Tween-20. The membranes were washed three times with TBS-T and imaged using Odyssey CLx imager.

Preparing Samples for Mass Spectrometry.

Photoaffinity samples were buffer exchanged to 100 mM ammonium bicarbonate (pH 8.0) using 10 kDa centrifugal filters. Samples were reduced with 12 mM dithiothreitol for 45 min at 56° C. and alkylated with 20 mM iodoacetic acid for 30 min at room temperature in the dark, followed by overnight digestion with a protease:protein ratio of 1:30 trypsin at 37° C. or 1:50 of chymotrypsin at 30° C. A final concentration of 1 mM CaCl₂) was added to samples digested with chymotrypsin. Digested peptides were cleaned using C18 zip tips according to manufacturer's instructions and dried via speedvac prior to storage in −20° C.

LC-MS/MS Analysis of Photoaffinity Labelled Samples.

Digested peptides were analyzed by LC-MS/MS using a LTQ Velos Orbitrap mass spectrometer. Peptides were eluted from a C18 pre-column (100 μM i.d.×2 cm) onto an analytical column (75 μM i.d.×2 cm). Solvent A was 0.1% FA. Solvent B (ACN, 0.1% FA) was applied as follows: 5-20% B over 75 min, 20-35% B over 25 min, 35% B for 19 min, 3 min ramp to 95% B and held for 18 min. Flow rates were 400 nL/min directed to an Advion NanoMate nano-ESI source held at 1.75 eV applied voltage.

Data-dependent scanning was performed with Xcalibur v 2.1.0 software using a survey mass scan at 60,000 resolution in the Orbitrap analyzer scanning mass/charge (m/z) range of 350-1600, followed by collision-induced dissociation tandem mass spectrometry (MS/MS) of the 6 most intense ions in the linear ion trap analyzer at 7,500 resolution. Precursor ions were selected by the monoisotopic precursor selection setting, with the instrument set to observe fragment ions once and then excluded from analysis for 45 seconds, allowing for interrogation of lower abundance ions. Ions were excluded with a ±10 ppm window. Precursor ions with a charge less than +3 were excluded from selection since ionization of IWP-854 increases the charge state of the peptide, making labeled peptides prone to higher charge states (+3 and above) and allowing for exclusion of peptides with a charge below +3.

Tandem mass spectra were searched against a protein database made by combining sequences for the sGC and H-NOX constructs analyzed with the proteome for Escherichia coli BL21, and common human contaminants. The combined database contained 5200 entries. MS/MS spectra were searched against the described protein database using Thermo Proteome Discovered 1.3, version 1.3.0.339 considering the tryptic peptides with up to 2 missed cleavage sites. Iodacetamide derivatives of cysteines and oxidation of methionines were specified as variable modifications. Modification by IWP-854 (1422.717 Da) was searched against all 20 amino acid residues in an iterative fashion. Proteins and peptides were visualized using the Discoverer software. IWP-854 modified residues were confirmed by the presence of a peak corresponding to the mass of the precursor ion minus the mass of 270.127 (FIG. 4). Peptides that were included in Table 1 were first identified automatically by the Discoverer software. Following initial identification, cross-linked peptides were also identified manually. Peptides identified manually displayed the correct m/z for the parent ion, loss of mass 270.127 during fragmentation as well as numerous identifying sequence ions, and the correct column retention time.

Molecular Modeling.

A molecular model for Ms sGC-NT13 was previously assembled using small-angle X-ray scattering (SAXS), chemical cross-linking and domain homology modeling. Models for compounds IWP-051 and IWP-864 were prepared by first generating a SMILES string in ChemDraw and submitting the string to the Grade Web Server (http://grade.globalphasing.org/cgi-bin/grade/server.cgi), which generates energy-minimized coordinates and refinement parameters using known structures for the Cambridge Structural Database. Modeling of compound binding was done manually in COOT followed by energy minimization in REFMAC5 as encoded in CCP4i. Structure figures were prepared using the Discovery Studio Visualizer 4.1 and PyMOL Molecular Graphics System, Version 1.8 Schrödinger, LLC.

TABLE 1
Summary of Residues Multiply Modified by IWP-854

| | | | |
|---|---|---|---|
| Modified Residues^{a} | Construct | Occurance^{b} | Error^{c} |

H-NOX Domain

| | | | |
|---|---|---|---|
| β1 6-8 | Ms sGC-NT^{d} | 18/26 | 3.2 ± 2.0 |
| | Sw H-NOX | 2/2 | 0.8 ± 0.8 |
| | Cb SONO | 3/3 | 1.78 |
| β1 41-48 | Hs sGC | 1/2 | 1.4 |
| | So H-NOX | 4/4 | 1.5 ± 2.3 |
| | Ns H-NOX | 3/3 | 0.4 ± 0.1 |
| | Sw H-NOX | 2/2 | 2.1 ± 0.4 |
| β1 48-51 | Hs sGC | 2/2 | 2.1 ± 1.0 |
| | Ns H-NOX | 3/3 | 1.1 ± 0.7 |
| β1 76-82 | Ns H-NOX | 2/3 | 1.3 ± 1.0 |
| | Sw H-NOX | 2/2 | 3.5 ± 0.1 |
| | Cb SONO | 3/3 | 1.07 |

Linker (H-NOX Domain - PAS Domain)

| | | | |
|---|---|---|---|
| β1 195-198 | Ms sGC-NT^{d} | 11/26 | 2.4 ± 1.0 |

Linker (PAS Domain - CC Domain)

| | | | |
|---|---|---|---|
| β1 328-331 | Ms sGC-NT^{d} | 11/26 | 3.1 ± 1.7 |

CC Domain

| | | | |
|---|---|---|---|
| β1 361-362 | Ms SGC-NT^{d} | 26/26 | 2.6 ± 1.7 |
| β1 365-366 | Ms sGC-NT^{d} | 26/26 | 2.6 ± 1.7 |
| β1 370-371 | Hs sGC | 2/2 | 1.4 ± 0.3 |
| β1 374-375 | Hs sGC | 2/2 | 1.4 ± 0.3 |
| β1 385 | Hs sGC | 2/2 | 2.2 ± 2.8 |

^{a}Included are peptides and sequence regions modified in more than one species. Modified residues are listed where known. A range of residues is listed where the exact modified residue could not be determined due to incomplete fragmentation. All peptides were in either the +3 or +4 charge states and had masses between 2200 and 3900 Da. Complete mass and charge information can be found in Table 2.
^{b}The number of times a peptide was identified out of the total number of experiments conducted.
^{c}Errors listed are the average and standard deviation of mass discrepancies for all peptides identified. For n = 2, the range is presented, and for n = 1, the single value is listed for ΔM.
^{d}For Ms sGC-NT experiments, results for all constructs and ligation states (±NO, CO, etc.) are combined. In each case, the modified peptide was identified in all Ms sGC-NT constructs analyzed (Ms sGC-NT23, Ms sGC-NT13, Ms sGC-β1).

Table 2: Detailed Summary of Peptides Modified by IWP-854.

Depicted is a summary of peptides modified by IWP-854 from Ms sGC-NT23, Ms sGC-NT13, Ms sGC β1 (1-380), Hs sGC, Ns H-NOX, So H-NOX, Sw H-NOX, and Cb SONO. Samples were photoaffinity labeled with IWP-854 and digested with trypsin or chymotrypsin as indicted. Digested samples were then analyzed by LC-MS/MS with both rounds of MS measured in high resolution mode. Peptides labelled by IWP-854 were identified both manually and using the Discoverer program

TABLE 2

| | | | | | | | |
|---|---|---|---|---|---|---|---|
| | Modified | Modified | | | Mass | | Error |
| Construct | Peptide | Residues^{a} | n^{b} | X_{corr} | (Da)^{c} | Charge | [ppm]^{d} |

H-NOX Domain

| | | | | | | | |
|---|---|---|---|---|---|---|---|
| Ms sGC-NT^{e} | β1 1-15 | N6, Y7, A8 | 18/26 | 4.19 | 3213.67 | +3, +4 | 3.2 ± 2.0 |
| (Trypsin) | | | | | 3229.65 | | |
| | | | | | 3245.64 | | |
| Hs sGC | α1 42-53 | P45-C47 | 1/2 | 0.97 | 2736.40 | +4 | 1.4 |
| (Trypsin) | β1 41-47 | I41-D44 | 1/2 | 0.57 | 2276.15 | +3 | 1.4 |
| | β1 48-57 | T48-L51 | 2/2 | 0.97 | 2461.27 | +4 | 2.1 ± 1.0 |
| So H-NOX | 41-57 | Y42-E46 | 4/4 | 4.35 | 3364.68 | +3, +4 | 1.5 ± 2.3 |
| (Trypsin) | | | | | | | |
| Ns H-NOX | 38-43 | M40-Y43 | 2/3 | 0.51 | 2092.01 | +3 | 0.9 ± 0.5 |
| (Chymotrypsin) | 38-49 | S44-D46 | 2/3 | 2.05 | 2772.27 | +3, +4 | 0.4 ± 0.1 |
| | | | | | 2788.27 | | 0.3 |
| | 44-49 | S44-D46 | 3/3 | 1.05 | 2122.00 | +3 | 0.7 ± 0.7 |
| | 44-59 | S44-D46 | 2/3 | 1.65 | 3098.54 | +4 | 0.2 |
| | 50-59 | H50-V52 | 2/3 | 1.24 | 2418.27 | +3, +4 | 0.7 ± 0.4 |
| | 50-66 | H50-V52 | 2/3 | 3.58 | 3142.65 | +3, +4 | 1.1 ± 0.7 |
| | 71-74 | G71-E72 | 2/3 | 0.92 | 1976.94 | +3 | 1.0 ± 0.7 |
| | 78-86 | S79-G84 | 2/3 | 1.75 | 2407.14 | +3 | 1.3 ± 1.0 |
| | 78-87 | S79-G84 | 2/3 | 0.96 | 2520.21 | +3, +4 | 1.1 ± 0.7 |
| | 168-177 | D173-D175 | 3/3 | 0.80 | 2641.25 | +3, +4 | 0.8 ± 0.5 |
| | 182-187 | E182-A184 | 3/3 | 0.64 | 2218.07 | +3 | 0.3 ± 0.3 |
| Cb SONO | 2-10 | T7 | 3/3 | 0.82 | 2383.27 | +3 | 1.78 |
| (Trypsin) | 76-95 | A76-Y81 | 3/3 | 1.76 | 3888.86 | +3 | 0.78 |
| | 176-181 | N176-Y177 | 3/3 | 0.49 | 2237.13 | +3 | 1.07 |
| Sw H-NOX | 6-15 | T6-G7 | 1/2 | 1.00 | 2086.05 | +3 | 0.8* |
| (Chymotrypsin) | 22-30 | S28-L30 | 1/2 | 1.97 | 2445.18 | +3 | 2.27* |
| | 26-36 | S28-L30 | 1/2 | 0.92 | 2680.24 | +3 | 3.04* |
| | 31-42 | I35-Y36 | 1/2 | 1.89 | 2713.30 | +3 | 1.89* |
| | 37-47 | E46-L48 | 1/2 | 0.99 | 2658.26 | +3, +4 | 2.1 ± 0.4 |
| | 37-48 | E46-L48 | 1/2 | 0.83 | 2771.35 | +3, +4 | 2.7 ± 0.8 |
| | 43-48 | E46-L48 | 2/2 | 0.46 | 2177.09 | +3 | 1.1 ± 0.1 |
| | 49-65 | P61-L65 | 2/2 | 2.02 | 3249.76 | +3, +4 | 1.8 ± 1.7 |
| | 51-65 | P61-L65 | 1/2 | 1.99 | 3008.61 | +3, +4 | 2.7 ± 0.7 |
| | 55-65 | E56-V57 | 1/2 | 2.59 | 2569.30 | +3, +4 | 2.5 ± 1.2 |
| | 55-69 | E56-V57 | 1/2 | 2.46 | 3084.62 | +4 | 2.2 |
| | 74-92 | V75-L77 | 1/2 | 2.45 | 3627.76 | +4, +5 | 3.5 ± 0.1 |
| | 96-107 | E105-Y107 | 1/2 | 2.18 | 2859.45 | +4 | 1.95 |
| | 146-152 | L146 | 1/2 | 0.23 | 2193.04 | +3 | 6.14* |
| | 146-153 | E151 | 1/2 | 0.30 | 2340.10 | +3 | 7.27* |
| | 157-174 | D167-E169 | 1/2 | 0.30 | 3361.64 | +4 | 1.2* |

H-NOX/PAS Linker

| | | | | | | | |
|---|---|---|---|---|---|---|---|
| Ms sGC-NT^{e} | β1 189-205 | A195-E198 | 11/26 | 2.45 | 3248.71 | +3, +4 | 2.4 ± 1.0 |
| (Trypsin) | | | | | | | |

PAS Domain

| | | | | | | | |
|---|---|---|---|---|---|---|---|
| Ms sGC-NT^{e} | β1 206-213 | F211-R213 | 5/26 | 0.60 | 2303.16 | +3, +4 | 3.2 ± 1.9 |
| (Trypsin) | | | | | | | |
| Ms sGC-NT23 | α1 279-286 | F284-K286 | 2/26 | 0.52 | 2247.15 | +3 | 0.1 ± 0.0 |
| (Trypsin) | | | | | | | |

PAS/Coiled-Coil Linker

| | | | | | | | |
|---|---|---|---|---|---|---|---|
| Ms sGC-NT^{e} | β1 328-341 | G328-I331 | 11/26 | 2.83 | 2917.51 | +3, +4 | 3.1 ± 1.7 |
| (Trypsin) | | | | | | | |

Coiled-Coil

| | | | | | | | |
|---|---|---|---|---|---|---|---|
| Ms sGC-NT^{e} | β1 356-366 | E361, V362 | 26/26 | 1.63 | 2696.43 | +3, +4 | 2.6 ± 1.7 |
| (Trypsin) | | D365, K366 | | | | | |
| Ms sGC-β1 | β1 342-355 | E351-D353 | 2/26 | 1.66 | 3110.50 | +4 | 0.9 ± 2.5 |
| (Trypsin) | | | | | 3126.51 | | |
| Hs sGC | β1 375 | E370, I371 | 2/2 | 1.19 | 2753.44 | +3, +4 | 1.4 ± 0.3 |
| (Trypsin) | | D374, R375 | | | | | |
| | β1 382-387 | D385 | 2/2 | 0.39 | 2127.07 | +3 | 2.2 ± 2.8 |

Cyclase

| | | | | | | | |
|---|---|---|---|---|---|---|---|
| Hs sGC | α1 628-637 | C629 | 1/2 | 0.82 | 2561.25 | +4 | 1.14 |
| (Trypsin) | | | | | | | |

^{a}Residues modified by IWP-854 are listed where known. Where the exact modified residue cannot be determined due to incomplete fragmentation, a range of residues is listed.
^{b}The number of times a peptide was identified out of the total number of experiments conducted (n). For Ms sGC-NT experiments, results for all constructs and ligation states (±NO, CO, etc.) are combined.
^{c}All peptides were in either the +3 or +4 charge states and had masses between 2200 and 3900 Da.
^{d}Errors listed are the average and standard deviation of mass discrepancies for all peptides identified. For n = 2, the range is presented, and for n = 1, the single value is listed for ΔM.
^{e}Peptides that were modified by IWP-854 in all Ms sGC-NT constructs analyzed (Ms sGC-NT23, Ms sGC-NT13, Ms sGC β1).

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

1. A method of identifying a binding site of a protein, the protein comprising a crosslinking site, the method comprising:
a. reacting said protein with a crosslinking agent at the crosslinking site to form a crosslinked protein, wherein the crosslinking agent comprises a substituent configured to fragment in tandem mass spectrometry to yield a signature mass fragment;
b. cleaving said protein into two or more peptides, wherein at least one of the peptides is tagged by the crosslinking agent; and
c. analyzing the peptides by tandem mass spectrometry to detect the tagged peptide, wherein said tagged peptide fragments to yield the signature mass fragment, and wherein the signature mass fragment is detected to identify the tagged peptide;
wherein the crosslinking agent comprises (1) an agonist or antagonist that binds to the binding site, and (2) a reactive moiety that reacts with the crosslinking site on the protein, and
wherein identification of the tagged peptide indicates the crosslinking site and the binding site on the protein.

2. The method of claim 1, wherein the binding site is in a vicinity of the crosslinking site.

3. The method of claim 1, wherein the crosslinking site is at a first distance away from the binding site, the first distance being about the same as a second distance stretching between the agonist or antagonist to the reactive moiety on the crosslinking agent.

4. The method of claim 1, wherein the binding site is a receptor.

5. The method of claim 1, wherein the crosslinking agent comprises a photo-cleavable diazirine moiety which transforms into a reactive carbene radical upon UV irradiation, wherein the carbene radical reacts with the protein to form the crosslinked protein.

6. (canceled)

7. The method of claim 1, wherein a bond linking an amide functional group and an ether functional group is broken during the analysis of the tagged peptide, thereby forming the signature mass fragment having an m/z ratio value.

8. The method of claim 1, wherein the substituent configured to fragment is according to the following structure:

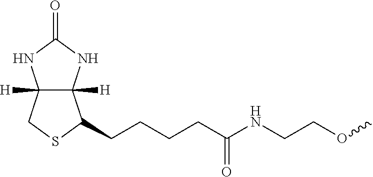

9. The method of claim 1, wherein the m/z ratio value of the signature mass fragment corresponds to an ion comprising the tagged peptide mass.

10. The method of claim 1, wherein the signature mass fragment corresponds to a singly charged fragment ion which comprises a biotin moiety with an amide substituent and has a m/z ratio of 270.127.

11. The method of claim 1, wherein the peptides are dissolved in acidic solvent before being analyzed by tandem mass spectrometry.

12. The method of claim 1, wherein the crosslinking agent is according to the following structure:

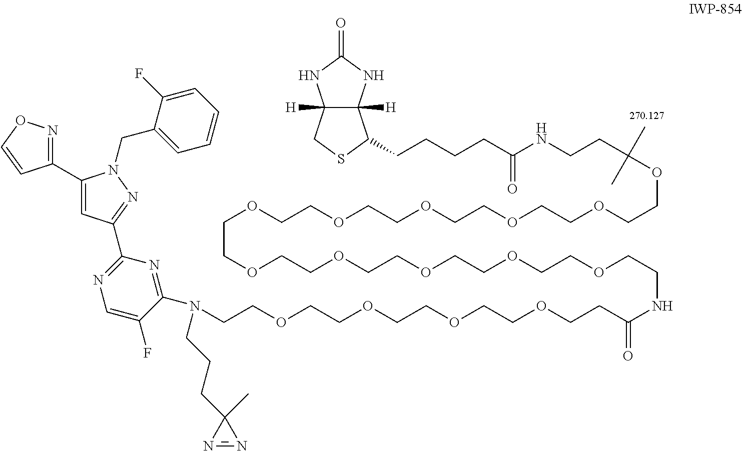

13. A method of identifying a binding site on a protein, wherein the method comprises:
a. reacting said protein with a crosslinking agent at a crosslinking site in a vicinity of the binding site, to form a crosslinked protein, wherein the crosslinking agent comprises a substituent configured to fragment in tandem mass spectrometry to yield a signature mass fragment;
b. cleaving said protein into two or more peptides, wherein at least one of the peptides is tagged by the crosslinking agent; and
c. analyzing the peptides by tandem mass spectrometry to detect the tagged peptide, wherein said tagged peptide fragments to yield the signature mass fragment, and wherein the signature mass fragment is detected to identify the tagged peptide;
wherein identification of the tagged peptide indicates the crosslinking site on the protein, and wherein identification of the crosslinking site aids in identifying a location of the binding site in the protein.

14. The method of claim 13, wherein the protein comprises a guanylyl or guanylate cyclase, a truncated version of guanylyl cyclase or a bacterial H-NOX homolog.

15.-16. (canceled)

17. The method of claim 13, wherein the binding site is a drug binding site and the crosslinking agent comprises a modified drug.

18.-24. (canceled)

25. A method of identifying a binding site on a peptide or protein in a complex mixture, wherein identification of the binding site allows for identification of the peptide or protein which comprises the binding site, wherein the method comprises;
a. providing the complex mixture;
b. introducing a crosslinking agent which selectively interacts with the peptide or protein which comprises the binding site, and reacts with the peptide or protein to form a tagged peptide or protein, wherein the crosslinking agent comprises a substituent configured to fragment in tandem mass spectrometry to yield a signature mass fragment; and
c. analyzing a portion of the complex mixture by tandem mass spectrometry to detect the tagged peptide or protein, wherein said tagged peptide or protein fragments to yield the signature mass fragment, and wherein the signature mass fragment is detected to identify the tagged peptide or protein.

26. The method of claim 25, wherein the complex mixture comprises at least one of the following: a peptide, protein, biomolecule, biopolymer, cell, cell lysate, pharmaceutical agent, DNA strand, organelle, or small-molecule.

27. The method of claim 25, wherein the crosslinking agent is configured to interact with said binding site.

28. The method of claim 27, wherein the binding site is a receptor and the crosslinking agent comprises an agonist or antagonist of said receptor.

29. The method of claim 27, wherein the binding site is a drug binding site and the crosslinking agent comprises a modified drug.

30. The method of claim 25, wherein the crosslinking agent comprises a photo-cleavable diazirine moiety which transforms into a reactive carbene radical upon UV irradiation, wherein the carbene radical reacts with the peptide or protein to form the tagged peptide or protein.

31.-36. (canceled)
